(19) United States
(12) Patent Application Publication
(10) Pub. No.: US 2023/0201397 A1
(43) Pub. Date: Jun. 29, 2023

(54) HEALTH SYSTEM OF FARM AND CONSTRUCTION EQUIPMENT

(71) Applicant: CNH Industrial Canada, Ltd., Saskatoon (CA)

(72) Inventor: Martin J. Roberge, Saskatoon (CA)

(21) Appl. No.: 17/562,673

(22) Filed: Dec. 27, 2021

(51) Int. Cl. A61L 2/26; B60R 25/31; B60R 25/01; B60R 25/24; B60S 1/64; G07C 5/08; A61L 2/04; A61L 2/10; A61L 2/22; A61L 2/24; A61B 5/01

(52) U.S. Cl. CPC A61L 2/26; A61B 5/01; A61L 2/04; A61L 2/10; A61L 2/22; A61L 2/24; B60R 25/01; B60R 25/24; B60R 25/31; B60S 1/64; G07C 5/085; A61B 2503/22; A61L 2202/11; A61L 2202/14; A61L 2202/17; A61L 2202/25

(57) ABSTRACT

A health system for a work vehicle includes a controller having a memory and a processor. The controller is configured to receive a first sensor signal indicative of a body temperature of an operator positioned outside a cab of the work vehicle. The controller is also configured to determine whether the body temperature of the operator is above a threshold body temperature. Furthermore, the controller is configured to output a first control signal to an interlock system indicative of instructions to lock a door of the cab of the work vehicle in response to determining the body temperature of the operator is above the threshold body temperature.

### BACKGROUND OF THE INVENTION

The present disclosure relates generally to a health system for a work vehicle.

Certain work vehicles, such as tractors or other prime movers, may be shared among multiple operators who may each use the work vehicle for several hours during the day. The operators may carry debris, germs, or undesired particles with them into the work vehicle during their shift. As such, the work vehicle may be manually sanitized before a new operator enters the work vehicle to prevent the spread of debris, germs, or undesired particles. Unfortunately, the process of manually sanitizing the work vehicle may be time-consuming to perform and complex/difficult to track/log.

### SUMMARY OF THE INVENTION

In certain embodiments, a health system for a work vehicle includes a controller having a memory and a processor. The controller is configured to receive a first sensor signal indicative of a body temperature of an operator positioned outside a cab of the work vehicle. The controller is also configured to determine whether the body temperature of the operator is above a threshold body temperature. Furthermore, the controller is configured to output a first control signal to an interlock system indicative of instructions to lock a door of the cab of the work vehicle in response to determining the body temperature of the operator is above the threshold body temperature

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a perspective view of an embodiment of a work vehicle including an embodiment of a health system;

FIG. 2 is a perspective view of an embodiment of an interior of the work vehicle of FIG. 1, including a portion of an embodiment of a health system;

FIG. 3 is a block diagram of an embodiment of a health system that may be employed with the work vehicle of FIG. 1;

FIG. 4 is a flow diagram of an embodiment of a method for sanitizing a work vehicle;

FIG. 5 is a flow diagram of an embodiment of a method for sanitizing a work vehicle after an operator change;

FIG. 6 is a flow diagram of an embodiment of a method for determining a sanitation mode for sanitizing a work vehicle; and

FIG. 7 is a flow diagram of an embodiment of a method for performing a manually initiated sanitation operation for a work vehicle.

### DETAILED DESCRIPTION

One or more specific embodiments of the present disclosure will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers’ specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements. Any examples of operating parameters and/or environmental conditions are not exclusive of other parameters/conditions of the disclosed embodiments.

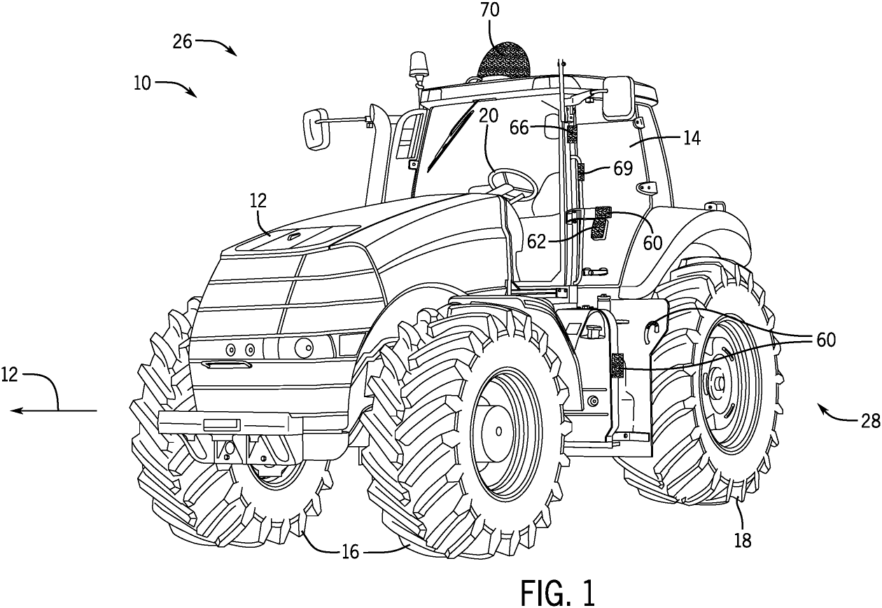

FIG. 1 is a perspective view of an embodiment of a work vehicle 10 including an embodiment of a health system. In the illustrated embodiment, the work vehicle 10 has a body 12 configured to house an engine (e.g., diesel engine), transmission, cooling system, and hydraulic system. Furthermore, the work vehicle 10 has a cab 14 where the operator may sit or stand to operate the work vehicle 10. The work vehicle 10 also has two front wheels 16 and two rear wheels 18 that rotate to move the work vehicle 10 through a field. The front wheels 16 and/or the rear wheels 18 may be driven to rotate by the engine and/or the hydraulic system (e.g., hydraulic motor(s) of the hydraulic system). While the illustrated work vehicle 10 includes wheels, in other embodiments, the work vehicle 10 may include tracks or a combination of wheels and tracks.

In the illustrated embodiment, the work vehicle 10 includes a steering wheel 20 within the cab 14. The steering wheel 20 is configured to control rotation of the front wheels 16 and/or the rear wheels 18, thereby controlling the direction of travel 12 of the work vehicle 10.

In the illustrated embodiment, the work vehicle 10 includes a health system 26 having a sanitation system 28. The sanitation system 28 includes cleaning devices 60 configured to perform an exterior sanitation on the work vehicle 10 (e.g., after an operator leaves the work vehicle 10). For example, the cleaning devices 60 of the sanitation system 28 may perform a dry sanitation and/or a wet sanitation to clean one or more handles on the work vehicle 10 after the operator departs. The health system 26 may perform the exterior sanitation on the work vehicle 10 immediately after the operator leaves the work vehicle 10. That is, the health system 26 may not lock the doors of the work vehicle 10 before initiating the exterior sanitation to clean one or more handles on the work vehicle 10. The health system 26 may lock the doors of the work vehicle 10 before performing the exterior sanitation on the work vehicle 10. The health system 26 also includes an operator ID (e.g., identification) device 62 (e.g., including a keypad, a card reader, etc.) configured to receive an operator ID. For example, the operator may enter the operator ID into a keypad of the operator ID device 62, a scanner of the operator ID device 62 may scan a token having an encoded operator ID, or a card reader of the operator ID device 62 may read a card with an encoded operator ID. In response to receiving the operator ID, the health system 26 may unlock a door of the cab 14 of the work vehicle 10, thereby enabling the operator to enter the cab 14. Furthermore, in certain embodiments, the health system 26 may save a record log, which includes the operator ID.

In certain embodiments, the health system 26 includes a body temperature scanner 64, which is configured to monitor a body temperature of an operator positioned outside the work vehicle cab 14. A controller of the health system 26 may determine whether the body temperature of the operator is above a threshold body temperature based on feedback from the body temperature scanner 64. In response to determining the body temperature of the operator is above the threshold body temperature, the controller may output a control signal to an interlock system 66 of the health system 26 indicative of instructions to lock the door of the cab, thereby blocking operator ingress into the cab. For example, the threshold body temperature may be a temperature corresponding to a fever. As such, a sick operator with a fever may not be able to access the work vehicle cab 14.

In certain embodiments, the controller of the health system 26 is configured to determine a work vehicle status based on received signals from the cleaning devices 60, the operator ID device 62, the body temperature scanner 64, the interlock system 66, or a combination thereof. The work vehicle status may include a standby mode, an operational mode, and a sanitation mode. In some embodiments, the standby mode represents the work vehicle 10 being shut down for the night or being unattended between operator shifts. For example, the work vehicle 10 may enter the standby mode at the end of the day, and the controller may instruct the interlock system 66 to keep the door locked. Furthermore, in certain embodiments, the work vehicle 10 enters standby mode every time the operator departs, and the controller may instruct the interlock system 66 to lock the door in response to the operator departing. In addition, the operational mode may represent that the work vehicle 10 is in use by the operator. For example, the work vehicle 10 may enter the operational mode (e.g., in which the controller instructs the interlock system to unlock the doors) if certain predetermined criteria are met (e.g., body temperature below the body temperature threshold, operator ID is same as a previous operator ID, etc.). Furthermore, the sanitation mode may be active immediately before, during, or immediately after a sanitation of the work vehicle 10. For example, the controller of the health system 26, while the work vehicle 10 is in the sanitation mode, may direct the cleaning devices 60 to perform a desired sanitation operation.

In an embodiment, the controller of the health system 26 may not activate the sanitation system 28 if the operator is positioned proximate to the exterior of the work vehicle 10. For example, the operator may enter the operator ID into a keypad of the operator ID device 62, indicating the operator presence near the work vehicle 10. As such, the controller of the health system 26 may not activate the sanitation system 28. In another example, the body temperature scanner 64 is configured to monitor a body temperature of an operator positioned outside the work vehicle 10. The body temperature scanner 64 may receive a body temperature of the operator, which is indicative of operator presence outside the work vehicle 10. As such, the controller of the health system 26 may not activate the sanitation system 28 to prevent harm to the operator. That is, the sanitation system 28 may not be initiated if the operator presence is detected proximate to the work vehicle 10.

The controller of the health system 26 is configured to output a signal to an indicator 70 indicative of instructions to display a light signal, output an audio signal, or a combination thereof. For example, the controller may be configured to instruct the indicator 70 to display a light to provide visual feedback to operators indicative of the vehicle status. In addition, the controller may be configured to instruct the indicator 70 to display a light and output the audio signal to indicate the work vehicle status. In the illustrated embodiment, the work vehicle 10 is a tractor. However, the health system 26 disclosed herein may be employed within other work vehicles, such as off-road vehicles (e.g., skids steers, construction equipment, etc.), agricultural vehicles (e.g., harvesters, sprayers, etc.), or any other suitable work vehicle types.

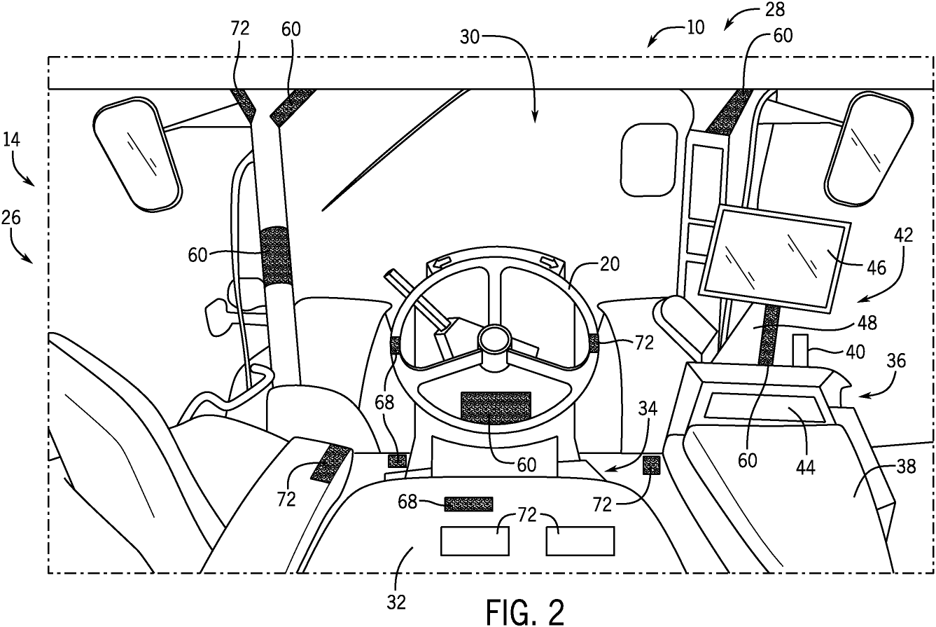

FIG. 2 is a perspective view of an embodiment of an interior 30 of the cab 14 of the work vehicle 10 of FIG. 1 (e.g., from the operator’s point of view), including a portion of an embodiment of a health system 26. As illustrated, the cab 14 includes a seat 32, on which the operator may sit while operating the work vehicle 10. In the illustrated embodiment, the steering wheel 20 is located near the seat 32, so as to be within reach of the operator while the operator is seated on the seat 32. Though a steering wheel 20 is included in the illustrated embodiment, other embodiments of the work vehicle 10 may include other and/or additional suitable devices for receiving steering inputs from the operator. For example, in certain embodiments, the work vehicle 10 may include left/right control bars, a hand controller, pedals, another suitable device for receiving steering inputs, or a combination thereof. In the illustrated embodiment, the work vehicle 10 includes pedals 34 configured to be actuated by feet of the operator. The pedals 34 may be configured to receive input from the operator for controlling the speed of the work vehicle 10. For example, the pedals 34 may control a throttle, brakes, a clutch, other suitable systems, or a combination thereof. In other embodiments, the pedals 34 may be used for steering inputs. Further, in certain embodiments, the pedals 34 may be omitted (e.g., in embodiments in which the speed control inputs are provided via a hand controller, etc.).

In the illustrated embodiment, an armrest assembly 36 is positioned along a lateral side of the seat 32. The armrest assembly 36 includes an armrest 38, a hand controller 40, and a user interface 42. The user interface 42 includes an input assembly 44 (e.g., including one or more buttons, one or more switches, one or more knobs, etc.), a display 46 (e.g., touchscreen display), and a support 48 configured to position the display 46 above the armrest 38. The display 46 may be used to present information to the operator, such as vehicle information (e.g., ground speed, oil pressure, engine temperature, etc.) and sanitation information (e.g., sanitation mode, operator ID, body temperature, record log, etc.). The hand controller 40 and the input assembly 44 may enable the operator to control the work vehicle 10 by inputting commands or instructions, which may be presented on the display 46. In certain embodiments, the display 46 may be a touchscreen display configured to enable the operator to input information by depressing buttons or other objects on the display 46. By navigating menus/controls on the display 46, the operator may control various features of the work vehicle 10 and/or an implement coupled to or towed by the work vehicle 10.

In the illustrated embodiment, the health system 26 includes one or more sensors 68 disposed within the cab 14, in which each sensor is configured to output a sensor signal indicative of operator presence within the cab 14. The sensor(s) 68 (e.g., body pressure sensor(s), temperature sensor(s), motion sensor(s), camera(s), infrared sensor(s), etc.) may be embedded in or placed on the steering wheel 20, the seat 32, the door, the floor, the roof, or a combination thereof, of the cab 14. Each sensor 68 is configured to output a sensor signal to the controller of the health system 26, which is configured to determine whether the operator is present within the cab 14. For example, sensor(s) 68 disposed in the door may output sensor signal(s) indicative of whether the door is locked or unlocked, and/or opened or closed, thereby enabling the controller to determine whether the operator is present within the cab 14 based on the state of the door. If the door is closed and locked, and the work vehicle 10 is in operation, the controller may determine the operator is present within the cab 14. Furthermore, sensor(s) 68 disposed on the seat 32 may output sensor signal(s) indicative of a weight on the seat, and the controller may be configured to determine operator presence within the cab 14 based on the measured weight.

In certain embodiments, if the controller determines the door is closed and locked, the operator is present within the cab, and a signal indicative of activation of the sanitation system 28 is received (e.g., from a user interface within the cab 14, from a remote system, etc.), the controller may not activate the sanitation system 28, and the controller may output a control signal to the indicator 70 indicative of instructions to activate. Furthermore, in certain embodiments, if the controller determines the door is closed and locked, and the operator is not present within the cab, the controller may output a control signal to the sanitation system 28 indicative of instructions to initiate a sanitation mode. For example, the controller of the health system (e.g., while operating in an automatic mode) may initiate a routine sanitization operation every night or at another suitable interval. The controller may verify that the operator is not present within the cab, and that the door is closed and locked before initiating sanitation operations. The controller may then output a control signal to cleaning devices 60 of the sanitation system 28 indicative of instructions to sanitize the interior 30 of the cab 14 of the work vehicle 10. The cleaning devices may include any suitable type(s) of device(s) configured to clean the work vehicle. For example, the cleaning devices 60 may include spray nozzle(s) configured to spray water (e.g., during a pre-rinse cycle, during a rinse cycle, etc.), spray nozzle(s) configured to spray wet sanitation fluid (e.g., during a clean cycle), air dryer(s) configured to dry the cleaned surfaces (e.g., during a dry cycle), motorized brush(es), other suitable device(s), or a combination thereof.

In the illustrated embodiment, the health system 26 also has one or more contamination sensors 72 disposed within the cab 14 and configured to monitor a level of contamination. The contamination sensor(s) 72 may include optical sensor(s), infrared sensor(s), friction sensor(s), any other sensor(s) capable of detecting dust, debris, bacteria, viruses, or a combination thereof. Each contamination sensor 72 is configured to output a signal indicative of the contamination level to the controller. In certain embodiments, the controller is configured to determine whether sanitation operations are to be performed for the entire work vehicle 10 or for one or more zones of the work vehicle 10 (e.g., the interior of the cab, the steering wheel 20, the door handle, the floor of the cab, etc.) based on feedback from the contamination sensor(s). For example, limiting sanitation operations to certain zone(s) may enhance the longevity of the components being sanitized. In addition, the duration associated with sanitizing one or more zones may be less than the duration associated with sanitizing the entire work vehicle 10. As such, it is time-efficient to sanitize one or more zones rather than the entire work vehicle 10. Furthermore, in certain embodiments, the controller may determine the sanitation mode (e.g., wet sanitation or dry sanitation) and/or the duration of sanitation operations based on feedback from the contamination sensor(s).

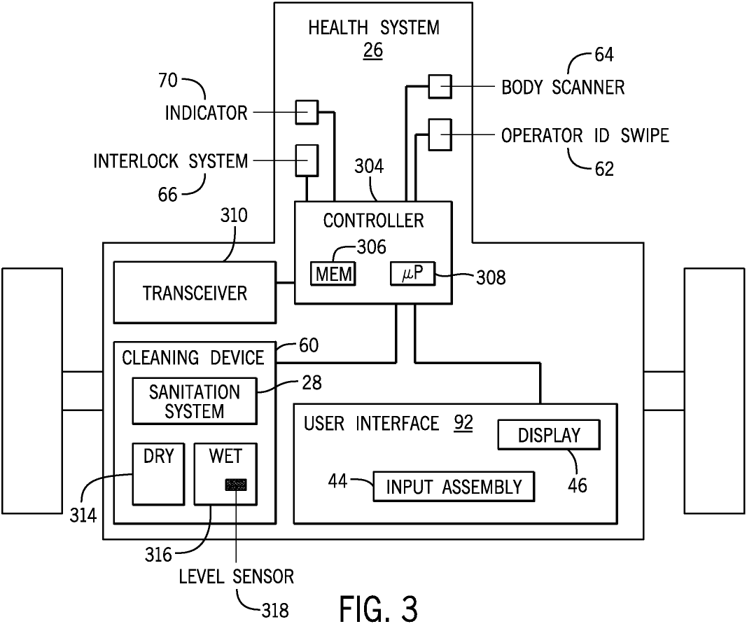

FIG. 3 is a block diagram of an embodiment of the health system 26 that may be employed with the work vehicle 10 of FIG. 1. In the illustrated embodiment, the health system 26 includes the sanitation system 28 having the cleaning device(s) 60, which are mounted to the work vehicle 10 and configured to perform sanitation operations. In the illustrated embodiment, the cleaning device(s) 60 include one or more dry sanitation devices 314 and one or more wet sanitation devices 316, which are mounted to the work vehicle 10 and configured to perform the sanitation. The dry sanitation devices 314 are configured to perform a heat sterilization, an ultra-violet (UV) light treatment, or a combination thereof. The wet sanitation devices 316 are configured to perform a pre-rinse cycle, a rinse cycle, a clean cycle, a dry cycle, or a combination thereof. In the illustrated embodiment, the sanitation system 28 includes a level sensor 318 (e.g., fluid level sensor) configured to output a signal to the controller 304 indicative of a wet sanitation fluid level. In certain embodiments, the controller 304 is configured to determine whether the wet sanitation fluid level is less than or equal to a threshold wet sanitation fluid level. For example, the threshold wet sanitation fluid level may correspond to a wet sanitation fluid level sufficient to complete wet sanitation operations. In response to determining the wet sanitation fluid level is less than the threshold wet sanitation fluid level, the controller 304 may output an information signal to the indicator 70 indicative of instructions to activate the indicator 70. While the sanitation system 28 includes the level sensor 318 in the illustrated embodiment, in other embodiments, the level sensor 318 may be omitted. Furthermore, while the sanitation system 28 includes wet and dry sanitation devices 314, 316 in the illustrated embodiment, in other embodiments, the sanitation system 28 may only include wet sanitation device(s) 316 or dry sanitation device(s) 314.

In the illustrated embodiment, the health system 26 also includes a controller 304, which is communicatively coupled to the user interface 42, the cleaning devices 60, the operator ID device 62, the body temperature scanner 64, the interlock system 66, the indicator 70, and a transceiver 310. The controller 304 is configured to output a control signal to the interlock system 66 indicative of instructions to lock and unlock the doors of the work vehicle 10, to output a control signal to the cleaning device(s) 60 indicative of instructions to initiate sanitation (e.g., without operator input, with limited operator input, etc.). In certain embodiments, the controller 304 is configured to output one or more signals to the transceiver 310, which in turn, outputs the signals to a remote system. In addition, the transceiver 310 is configured to receive signals from a remote system and to convey the signals to the controller 304.

In certain embodiments, the controller 304 is an electronic controller having electrical circuitry configured to process data from the user interface 42, the operator ID device 62, the body temperature scanner 64, the interlock system 66, the transceiver 310, or a combination thereof. In the illustrated embodiment, the controller 304 includes a processor, such as the illustrated microprocessor 308, and a memory device 306. The controller 304 may also include one or more storage devices and/or other suitable components. The processor 308 may be used to execute software, such as software for controlling the interlock system 66, software for controlling the cleaning devices 60, software for controlling the user interface 42, and so forth. Moreover, the processor 308 may include multiple microprocessors, one or more “general-purpose” microprocessors, one or more special-purpose microprocessors, and/or one or more application specific integrated circuits (ASICS), or some combination thereof. For example, the processor 308 may include one or more reduced instruction set (RISC) processors.

The memory device 306 may include a volatile memory, such as random access memory (RAM), and/or a nonvolatile memory, such as read-only memory (ROM). The memory device 306 may store a variety of information and may be used for various purposes. For example, the memory device 306 may store processor-executable instructions (e.g., firmware or software) for the processor 308 to execute, such as instructions for controlling the interlock system 66, instructions for controlling the cleaning devices 60, and so forth. The storage device(s) (e.g., nonvolatile storage) may include ROM, flash memory, a hard drive, or any other suitable optical, magnetic, or solid-state storage medium, or a combination thereof. The storage device(s) may store data (e.g., threshold values, a record log, etc.), instructions (e.g., software or firmware for controlling the cleaning devices, etc.), and any other suitable data.

In the illustrated embodiment, the sanitation system 28 includes the user interface 42, which is communicatively coupled to the controller 304. The user interface 42 is configured to enable the operator to control certain parameter(s) associated with operation of the work vehicle 10 and/or the health system 26. In the illustrated embodiment, the user interface 42 includes the input assembly 44 and the display 46. The display 46 is configured to present information to the operator, such as a graphical representation of the wet sanitation fluid level, a visual representation of the record log, a visual representation of certain parameter(s) associated with operation of the work vehicle (e.g., fuel level, oil pressure, water temperature, etc.), a visual representation of certain parameter(s) associated with the health system 26 (e.g., a time of day to initiate sanitation operations, a day of week to initiate sanitation operations, a sanitation duration, sanitation location(s)/zone(s), contamination level(s), one or more environmental factors, etc.), a visual representation of certain parameter(s) associated with operation of the sanitation system 28 (e.g., sanitation mode, wet sanitation fluid level, etc.), other suitable information, or a combination thereof. In certain embodiments, the display 46 may include a touchscreen interface that enables the operator to control certain parameters (e.g., sanitation mode, sanitation duration, etc.) associated with operation of the work vehicle 10 and/or the health system 26.

In the illustrated embodiment, the transceiver 310 is communicatively coupled to the controller 304. The transceiver 310 is configured to establish a communication link with a corresponding transceiver 310 of a remote system (e.g., a base station and/or another work vehicle), thereby facilitating communication between the remote system and the health system 26 of the work vehicle 10. The transceiver 310 may operate at any suitable frequency range within the electromagnetic spectrum. For example, in certain embodiments, the transceiver 310 may broadcast and receive radio waves within a frequency range of about 400 MHz to about 6 GHz. In addition, the transceiver 310 may utilize any suitable communication protocol, such as a standard protocol (e.g., Wi-Fi, Bluetooth, etc.) or a proprietary protocol. For example, the operator may use a remote system (e.g., smart phone, computer, tablet) to output signal(s) to the transceiver 310 indicative of instructions to enter a sanitation mode. In another example, the transceiver 310 may output the record log to the remote system for viewing.

The controller 304 of the health system 26 may be configured to receive various inputs (e.g., to control various parameters of the sanitation operation). The inputs may include one or more times of day to initiate sanitation operations, one or more days of the week to initiate sanitation operations, a sanitation duration, a sanitation mode (e.g., wet sanitation mode or dry sanitation mode), one or more zones of the cab 14 to be cleaned by the sanitation system 28, threshold level(s) of contamination, one or more environmental factors (e.g., quantity of dust within the field, quantity of mud within the field, etc.), or any combination thereof. For example, the operator may instruct the controller 304, via the display 46 or the input assembly 44, to automatically sanitize the work vehicle 10 every other day at the end of the work day. In another example, the operator may instruct the controller 304, via the display 46 or the input assembly 44, to perform dry sanitation on the weekdays and wet sanitation on the weekends (e.g., if the contamination is greater than the threshold level of contamination). In yet another example, the operator may instruct the controller 304, via the display 46 or the input assembly 44, to perform the sanitation in one or more zones of the cab 14.

In the illustrated embodiment, the controller 304 is configured to receive one or more signals from the operator ID device 62, the body temperature scanner 64, the interlock system 66, or a combination thereof. For example, in certain embodiments, the controller 304 is configured to receive a sensor signal from the body temperature scanner 64 indicative of a body temperature of the operator and to determine whether the body temperature of the operator is above a threshold body temperature. The threshold body temperature may be a temperature corresponding to a fever (e.g., 99° F./37.2° C., 100° F./37.8° C., 101° F./38.3° C., etc.). In certain embodiments, the controller 304, in response to determining the body temperature of the operator is above the threshold body temperature, may output a control signal to the interlock system 66 indicative of instructions to lock the door of the cab or maintain the locked state of the door, thereby blocking entry of the operator into the cab. Furthermore, in certain embodiments, the controller 304 may receive a sensor signal indicative of presence of the operator within the work vehicle 10, and the controller 304 may output a control signal to the interlock system 64 indicative of instructions to lock the door of the cab or maintain the locked state of the door and output a control signal to the sanitation system 28 indicative of instructions to enter a sanitation mode in response to determining the operator is not present within the cab of the work vehicle 10.

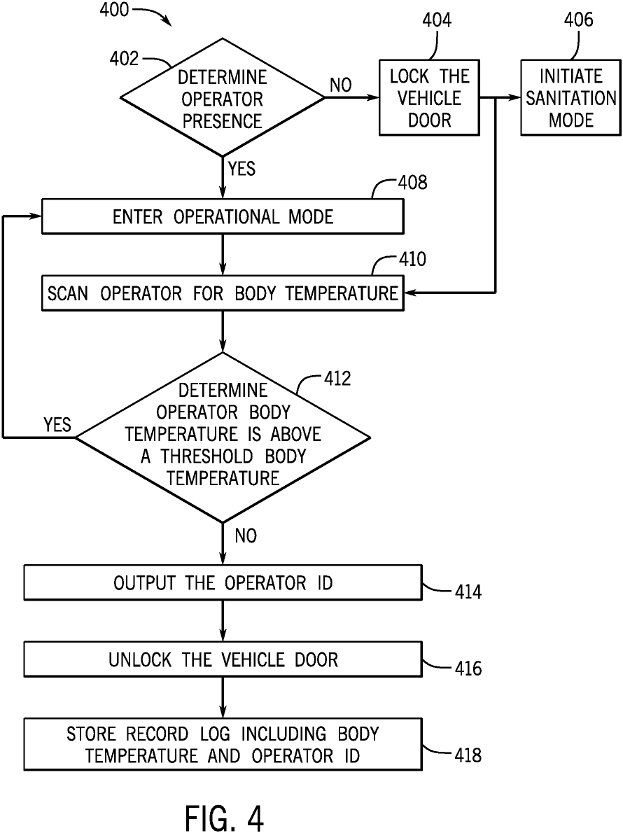

FIG. 4 is a flow diagram of an embodiment of a method 400 for sanitizing a work vehicle 10. First, as represented by block 402, a determination is made regarding whether an operator is present. For example, the controller may receive sensor signal(s) from the interlock system, the operator ID device, the body temperature scanner, the operator presence sensors, or a combination thereof, and determine the operator presence based on the sensor signal(s). If the operator is not present, the sanitation system may enter a standby mode or a sanitation mode. For example, the operator may leave the work vehicle to remove an obstacle or to take a lunch break. As such, the operator may instruct the sanitation system to enter a standby mode, and the health system may instruct the interlock system to lock the door of the cab of the work vehicle, as represented by block 404. Furthermore, the operator may instruct the sanitation system to enter a sanitation mode (e.g., via an indication that the operator is shutting down the work vehicle for the day). As such, the controller (e.g., of the health system) may output a control signal to the interlock system indicative of instructions to lock the door, as represented by block 404, and a control signal to the sanitation system indicative of instructions to enter a sanitation mode, as represented by block 406.

Furthermore, in certain embodiments, if the operator is not present, the controller may receive a sensor signal from the contamination sensor(s) indicative of the level of contamination. As such, the controller (e.g., of the health system) may output a control signal to the interlock system indicative of instructions to lock the door of the cab, as represented by block 404. Next, the controller (e.g., of the health system) may output a control signal to the sanitation system indicative of instructions to enter the sanitation mode, as represented by block 406. For example, in response to the controller determining the level of contamination is greater than a threshold level, the controller may instruct the sanitation system to enter the sanitation mode. The work vehicle may have multiple operators throughout the day. After the first operator departs, the controller may output the control signal to the interlock system indicative of instructions to lock the door of the cab, as represented by block 404, and output the control signal to the sanitation system indicative of instructions to enter the sanitation mode, as represented by block 406 (e.g., enter the dry sanitation mode to perform dry sanitation on the handles of the work vehicle, enter a wet sanitation mode to perform wet sanitation of interior component(s) of the cab, etc.). Furthermore, if the operator is present, the sanitation system may enter an operational mode (e.g., in which the work vehicle performs agricultural operations throughout the field), as represented by block 408.

As previously discussed, if the operator is not present within the cab of the work vehicle, the controller instructs the interlock system to lock the door of the cab, as represented by block 404. In certain embodiments, the controller (e.g., of the health system) outputs a control signal to the body temperature scanner indicative of instructions to initiate a body temperature scan, and the body temperature scanner scans the body temperature of the operator positioned outside of the work vehicle, as represented by block 410. In certain embodiments, the body temperature scan may be performed in response to manual operator activation (e.g., in response to the operator ID device receiving the operator ID, in response to the operator activating a control, etc.), and/or the body temperature scan may be performed in response to automatic activation (e.g., in response to detection of the operator proximate to the cab door, etc.). Next, as represented by block 412, a determination is made regarding whether the received body temperature of the operator is above the threshold body temperature.

The controller (e.g., of the health system) is configured to receive a sensor signal from the body temperature scanner indicative of the body temperature of the operator. If the body temperature of the operator is above the threshold body temperature, the controller outputs a control signal to the interlock system indicative of instructions to lock the door of the cab. As used herein, locking the door of the cab includes transitioning the door from an unlocked state to a locked state and maintaining the door in the locked state if the door is already locked. As previously discussed, the threshold body temperature may be a temperature corresponding to a fever. As such, a sick operator with a fever may not be able to access the work vehicle cab.

If the body temperature of the operator is less than or equal to the threshold body temperature, the controller (e.g., of the health system) may output a control signal to the operator ID device indicative of instructions to display (e.g., flash) a light, output an audio signal, or otherwise inform the operator that the operator ID device is ready to receive the operator ID. For example, the operator ID device may change a light signal from a red light to a green light as indication to the operator. Even if the operator has a higher than normal body temperature (e.g., from performing manual work within the field, etc.), the controller may determine the body temperature is less than or equal to the threshold body temperature because the threshold body temperature corresponds to a fever. Once the operator ID device indicates that the operator ID device is ready to receive the operator ID, the operator may input the operator ID into the operator ID device, and the operator ID device may output the operator ID to the controller, as represented by block 414.

In response to receiving the operator ID, the controller (e.g., of the health system) may output a control signal to the interlock system indicative of instructions to unlock the door of the work vehicle cab, as represented by block 416. For example, in certain embodiments, the controller may only unlock the door in response to the operator ID matching the operator ID of an authorized operator. In certain embodiments, the controller may also change the work vehicle status to the operational mode. In certain embodiments, the controller outputs the body temperature of the operator and the operator ID to the transceiver for transmission to a remote device. Additionally or alternatively, the controller may generate/store a record log that includes the body temperature of the operator and the operator ID, as represented by block 418. The record log may also contain sanitation data from the sanitation system, state(s) of the door from the interlock system, data from the operator presence sensor(s), data from the contamination sensor(s), the work vehicle status, other suitable parameters, or a combination thereof. The operator may be able to view the record log on the display or on the remote system (e.g., smart phone, computer, tablet). In certain embodiments, the operator may also verify, edit, or download the record log (e.g., via the display 46 or the remote system). The method described above may be stored on one or more tangible, non-transitory, machine-readable media and/or may be performed by the processor of the controller described above with reference to FIG. 3 or on another suitable controller. The steps of the method 400 may be performed in the order disclosed above or in any other suitable order. Furthermore, certain steps of the method may be omitted. For example, in certain embodiments, the step of receiving the operator ID may be omitted.

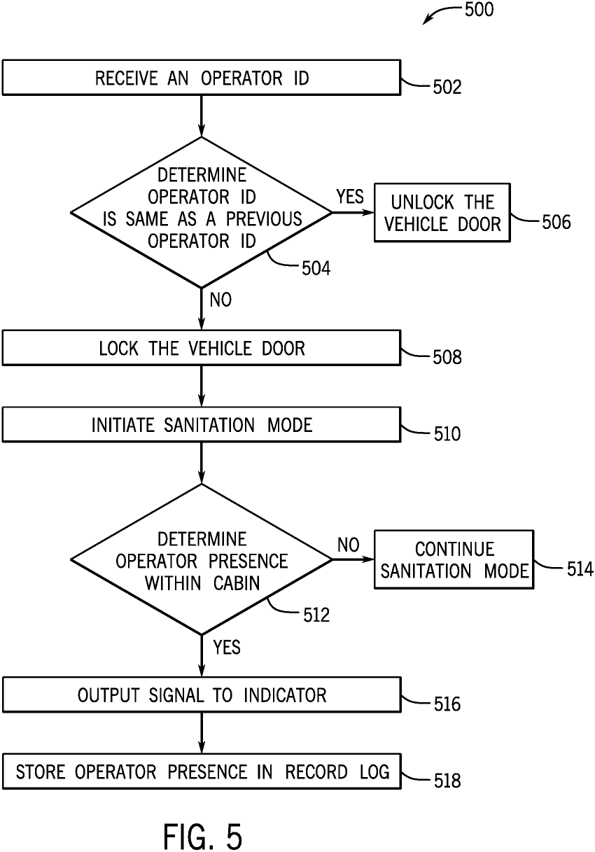

FIG. 5 is a flow diagram of an embodiment of a method 500 for sanitizing a work vehicle after an operator change. First, as represented by block 502, the controller (e.g., of the health system) receives an input signal from the operator ID device indicative of the operator ID. The controller may then compare the operator ID to the last operator ID entry on the record log to determine whether the operator IDs are the same, as represented by block 504. If the operator IDs are the same, the controller outputs a control signal to the interlock system indicative of instructions to unlock the doors of the work vehicle, as represented by block 506. Accordingly, sanitation is not performed on the work vehicle because no operator change occurred.

If the operator IDs are not the same, the controller (e.g., of the health system) outputs a control signal to the interlock system indicative of instructions to lock the door of the cab, as represented by block 508, thereby enabling the sanitation system to sanitize the work vehicle between operators. After the door is locked, the controller (e.g., of the health system) outputs a control signal to the sanitation system indicative of instructions to enter/initiate a sanitation mode (e.g., dry sanitation mode, wet sanitation mode, etc.), as represented by block 510. The control signal may be output to the cleaning devices of the sanitation system located outside the work vehicle, inside the cab, or a combination thereof. For example, the control signal may instruct the cleaning devices disposed outside the work vehicle to initiate the dry sanitation on the handles of the work vehicle. Additionally or alternatively, the control signal may instruct the cleaning devices disposed inside the cab to perform the dry sanitation and/or the wet sanitation.

As represented by block 512, the controller (e.g., of the health system) is configured to determine whether the operator is present within the cab. The dry sanitation may include the heat sterilization, the UV light treatment, or a combination thereof. In addition, the wet sanitation may include spraying cleaning solution onto various surfaces. As such, the controller is configured to verify that the operator is not present in the cab 14 before continuing sanitation. In certain embodiments, the controller may perform the operator presence determination at selected intervals (e.g., every second, every ten seconds, every minute, every ten minutes, etc.) during the sanitation process. For example, the operator presence sensor(s) may monitor the interior of the cab for the presence of the operator. In another example, the interlock system may monitor the state of the door (e.g., locked or unlocked) to enable the controller to determine operator presence.

If the controller (e.g., of the health system) determines no operator is present within the cab, the controller may enable the sanitation system to continue the sanitation operation, as represented by block 514. As such, the cleaning devices continue the sanitation operation. In certain embodiments, the controller stores sanitation data and/or generates the record log based on the sanitation data. In some embodiments, the controller analyzes the record log to determine if there are any gaps in the sanitation operations.

If the controller (e.g., of the health system) determines the operator is present within the cab, the controller outputs a control signal to the sanitation system indicative of termination of the sanitation operation, and the controller outputs a control signal to the indicator indicative of instructions to activate, as represented by block 516. For example, the indicator may flash a light and/or output an audio signal to indicate operator presence in response to receiving the control signal from the controller. With regard to sanitation operations outside the cab, in certain embodiments, the sanitation operations may continue while the operator is present within the cab.

If the indicator is activated, the controller (e.g., of the health system) may output a control signal to the transceiver indicative of instructions to output a signal to the remote system indicative of the operator presence within the cab. Additionally or alternatively, the controller may store an indication in the record log indicative of operator presence within the cab, as represented in block 518. In certain embodiments, the controller may also store the operator ID of the present operator within the record log. Furthermore, in certain embodiments, the controller may store additional information, such as the sanitation mode, the state(s) of the door, other suitable information, or a combination thereof, within the record log. The method described above may be stored on one or more tangible, non-transitory, machine-readable media and/or may be performed by the processor of the controller described above with reference to FIG. 3 or on another suitable controller. The steps of the method 500 may be performed in the order disclosed above or in any other suitable order. Furthermore, certain steps of the method may be omitted.

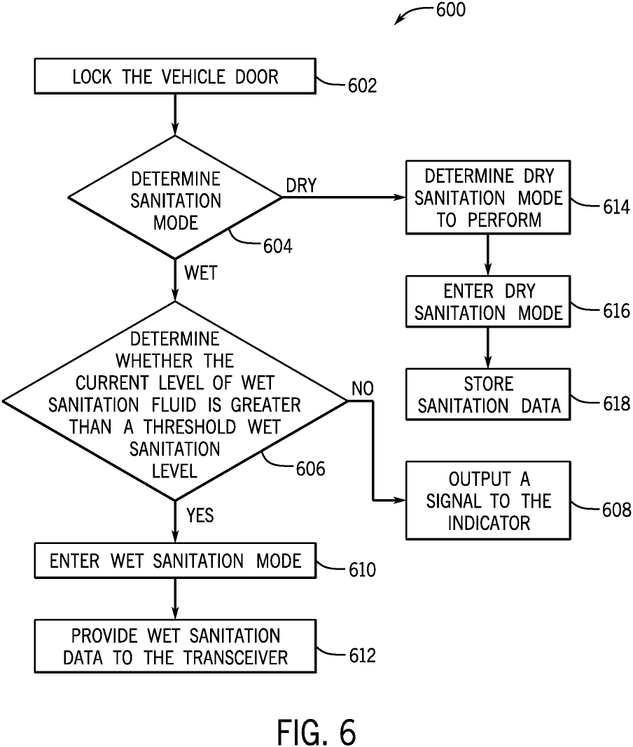

FIG. 6 is a flow diagram of an embodiment of a method 600 for selecting a sanitation mode for sanitizing the work vehicle. First, the controller (e.g., of the health system) outputs a control signal to the interlock system indicative of instructions to lock the door of the work vehicle, as represented by block 602. In certain embodiments, the controller may determine whether the operator is present within the cab before locking the door, as disclosed above. Next, the controller determines a sanitation mode from a set of candidate sanitation modes (e.g., wet sanitation mode and dry sanitation mode), as represented by block 604. The determination of the sanitation mode may be based on time of day, day of week, sanitation duration, sanitation location, a contamination level, one or more environmental factors, or a combination thereof. For example, once a week the controller may instruct the sanitation system to enter the wet sanitation mode to perform a more thorough cleaning. By way of further example, the controller may instruct the sanitation system to enter the wet sanitation mode in response to feedback from the contamination sensor(s) indicating a higher level of contamination. In addition, the controller may instruct the sanitation system to enter the wet sanitation mode at the end of the day and to enter the dry sanitation mode at all other times of day. Furthermore, if a longer sanitation duration is available, the controller may instruct the sanitation system to enter the wet sanitation mode, and if a shorter sanitation duration is available, the controller may instruct the sanitation system to enter the dry sanitation mode. In addition, the controller may instruct the sanitation system to enter the wet sanitation mode to sanitize certain zone(s) of the work vehicle, and the controller may instruct the sanitation system to enter the dry sanitation mode to sanitize other zone(s) of the work vehicle. If the environment of the field has an excessive amount of dirt and/or debris, the controller may instruct the sanitation system to enter the wet sanitation mode to provide a more thorough cleaning, and if the environment of the field has less dirt and/or debris, the controller may instruct the sanitation system to enter the dry sanitation mode to reduce usage of cleaning fluid. In certain embodiments, the operator may manually override the selected sanitation mode. While automatic selection of the sanitation mode is disclosed herein, in certain embodiments, the sanitation mode may be manually selected by the operator.

In response to selection of the wet sanitation mode, the controller (e.g., of the health system) is configured to initiate a wet sanitation process (e.g., enter wet sanitation mode). The controller may receive a sensor signal from a level sensor indicative of the current level of wet sanitation fluid present within the wet sanitation system. The controller may then determine whether the current level of wet sanitation fluid is greater than a threshold wet sanitation level (e.g., sufficient to complete the wet sanitation), as represented by block 606. For example, the wet sanitation mode may include the clean cycle, which utilizes the wet sanitation fluid. The wet sanitation mode may also include the pre-rinse cycle and the rinse cycle, which utilize water. Accordingly, in certain embodiments, the controller may also determine whether the level of water is greater than a threshold water level (e.g., based on feedback from water level sensor(s)).

If the current level of wet sanitation fluid is less than or equal to the threshold wet sanitation level, the controller (e.g., of the health system) outputs a signal to the indicator, as represented by block 608. For example, the controller may be configured to instruct the indicator to display a light and/or output an audio signal to indicate the low level of wet sanitation fluid, thereby providing an indication to the operator to add wet sanitation fluid. As the operator adds the wet sanitation fluid to the wet sanitation system, the level sensor may output a sensor signal to the controller indicative of a change in the wet sanitation fluid level. The process may then return to block 606, in which the controller determines whether the current level of wet sanitation fluid is greater than a threshold wet sanitation level.

If the current level of wet sanitation fluid is greater than the threshold level, the controller (e.g., of the health system) outputs a control signal to the sanitation system (e.g., the cleaning devices of the sanitation system) indicative of instructions to enter the wet sanitation mode (e.g., perform wet sanitation operations), as represented in block 610. In certain embodiments, the wet sanitation mode includes a pre-rinse cycle, a rinse cycle, a clean cycle, a dry cycle, or a combination thereof. For example, based on certain parameters (e.g., time of day, day of week, available time before next agricultural operation is scheduled, level of contamination, etc.), the controller may instruct the sanitation system to enter the wet sanitation mode by performing a subset of the cycles (e.g., only the clean cycle and the dry cycle). Alternatively, the controller may instruct the sanitation system to enter the wet sanitation mode by performing all of the cycles to provide a more thorough cleaning. In certain embodiments, the operator may manually override the number of cycles selected by the controller, and/or the operator may manually select the number of cycles. While a pre-rinse cycle, a rinse cycle, a clean cycle, a dry cycle are disclosed herein, in certain embodiments, the wet sanitation mode may include fewer cycles (e.g., a subset of the cycles disclosed above) or more cycles (e.g., including additional type(s) of cycle(s)). Furthermore, in certain embodiments, at least one cycle may be performed multiple times during the wet sanitation process.

After successful completion of the wet sanitation, the controller (e.g., of the health system) stores the sanitation data associated with the sanitation mode (e.g., time of sanitation, date of sanitation, cycle(s) performed, duration of sanitation, etc.) to the record log, as represented by block 612. As described above, the operator can view, verify, edit, or download the record log (e.g., on the display of the work vehicle and/or on a remote system). Furthermore, in certain embodiments, the controller may output the sanitation data to the transceiver, and the transceiver may output the sanitation data to a remote system. In certain embodiments, in response to completion of wet sanitation of the interior of the cab (e.g., or after a delay period sufficient to enable the water/wet sanitation fluid to dry after completion of wet sanitation), the controller may instruct the interlock system to unlock the door of the cab and/or enable the interlock system to unlock the door (e.g., in response to an operator providing an operator ID).

In response to selection of the dry sanitation mode, the controller (e.g., of the health system) may determine a type of dry sanitation to perform, as represented by block 614. For example, the controller may receive sensor signal(s) from the contamination sensor(s) indicative of a current contamination level (e.g., within the cab, on exterior surfaces, etc.). For example, optical contamination sensor(s) may monitor an amount of debris or particles. The controller may receive the sensor signal(s) and determine a type of dry sanitation to perform based on the sensor signal(s). In certain embodiments, the dry sanitation may include a heat sterilization, a UV light treatment, or a combination thereof. Accordingly, the controller may determine whether to perform heat sterilization, UV light treatment, other type(s) of dry sanitation, or a combination thereof. In some embodiments, the controller may determine the type of dry sanitation based on the surface(s) being sanitized. For example, treating certain surfaces with one type of dry sanitation may increase the longevity of the surface, as compared to treating the surface with another type of dry sanitation. Furthermore, in certain embodiments, the controller may select a degree of dry sanitation (e.g., duration of dry sanitation, intensity, etc.) based on the type of surface. While selection of the type of dry sanitation is disclosed above, in certain embodiments, the controller may apply the same type of dry sanitation for each operation of the dry sanitation mode.

The controller may output a control signal to the sanitation system indicative of instructions to enter the dry sanitation mode (e.g., initiate dry sanitation operations), as represented by block 616. For example, the controller may output the control signal to the cleaning device(s) to initiate UV treatment on the exterior handles of the work vehicle. In another example, the controller may output the control signal to the cleaning device(s) to initiate heat treatment for component(s) within the cab 14. After successful completion of the dry sanitation, the controller (e.g., of the health system) stores the sanitation data associated with the sanitation mode (e.g., time of sanitation, date of sanitation, type of dry sanitation, duration of sanitation, etc.) to the record log, as represented by block 618. As described above, the operator can view, verify, edit, or download the record log (e.g., on the display of the work vehicle and/or on a remote system). Furthermore, in certain embodiments, the controller may output the sanitation data to the transceiver, and the transceiver may output the sanitation data to a remote system. While a wet sanitation mode and a dry sanitation mode are disclosed above, in certain embodiments, the candidate sanitation modes may include other/additional type(s) sanitation mode(s), such as a combination of wet and dry sanitation. The method described above may be stored on one or more tangible, non-transitory, machine-readable media and/or may be performed by the processor of the controller described above with reference to FIG. 3 or on another suitable controller. The steps of the method 600 may be performed in the order disclosed above or in any other suitable order. Furthermore, certain steps of the method may be omitted.

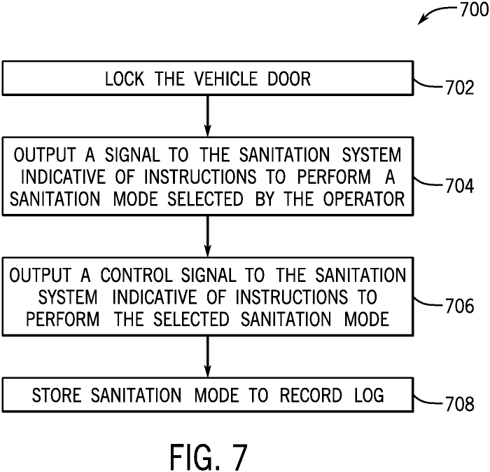

FIG. 7 is a flow diagram of an embodiment of a method 700 for performing a manually initiated sanitation operation for the work vehicle. First, as represented by block 702, the controller (e.g., of the health system) is configured to output a control signal to the interlock system indicative of instructions to lock the door of the cab. The controller may then output a control signal to the sanitation system indicative of instructions to perform a sanitation mode selected by the operator, as represented by block 704. For example, the operator may provide input to the controller (e.g., via the display within the cab, via the remote system, etc.) to perform a sanitation operation. The operator may select various parameters associated with the sanitation, such as the sanitation mode, the number/type of cycle(s) for wet sanitation, the duration of sanitation, the type of sanitation for dry sanitation, the time for initiating sanitation, etc.

In certain embodiments, the controller (e.g., of the health system) may output a control signal to the sanitation system indicative of instructions to perform the selected sanitation mode, as represented by block 706. After successful completion of the sanitation, the controller (e.g., of the health system) stores the sanitation data associated with the sanitation mode (e.g., time of sanitation, date of sanitation, cycle(s) performed, type of sanitation, duration of sanitation, etc.) to the record log, as represented by block 708. As described above, the operator can view, verify, edit, or download the record log (e.g., on the display of the work vehicle and/or on a remote system). Furthermore, in certain embodiments, the controller may output the sanitation data to the transceiver, and the transceiver may output the sanitation data to a remote system. The method described above may be stored on one or more tangible, non-transitory, machine-readable media and/or may be performed by the processor of the controller described above with reference to FIG. 3 or on another suitable controller. The steps of the method 700 may be performed in the order disclosed above or in any other suitable order. Furthermore, certain steps of the method may be omitted.

By way of further example, in certain embodiments, the controller (e.g., of the health system) may receive an input signal indicative of an operator ID (e.g., from the operator ID device), in which the operator ID corresponds to the operator of the work vehicle. The controller may also receive a sensor signal indicative of absence of the operator from the cab of the work vehicle (e.g., based on feedback from the operator presence sensor(s)). Furthermore, the controller may output a first control signal to the interlock system indicative of instructions to lock the door of the cab and output a second control signal to the sanitation system indicative of instructions to enter a sanitation mode in response to determining the operator is absent from the cab of the work vehicle. In addition, the controller is configured to save a record log, in which the record log includes the operator ID and the sanitation mode. In certain embodiments, the record log may also include a time of sanitation, a date of sanitation, a duration of sanitation, or any combination thereof.

While only certain features have been illustrated and described herein, many modifications and changes will occur to those skilled in the art. It is, therefore, to be understood that the appended claims are intended to cover all such modifications and changes as fall within the true spirit of the disclosure.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for (perform)ing (a function)... ” or “step for (perform)ing (a function)... ”, it is intended that such elements are to be interpreted under 35 U.S.C. 112(f). However, for any claims containing elements designated in any other manner, it is intended that such elements are not to be interpreted under 35 U.S.C. 112(f).

1. A health system for a work vehicle, comprising:
a controller comprising a memory and a processor, wherein the controller is configured to:
receive a first sensor signal indicative of a body temperature of an operator positioned outside a cab of the work vehicle;
determine whether the body temperature of the operator is above a threshold body temperature; and
output a first control signal to an interlock system indicative of instructions to lock a door of the cab in response to determining the body temperature of the operator is above the threshold body temperature.

2. The health system of claim 1, wherein the controller is configured to output a second control signal to the interlock system indicative of instructions to unlock the door of the cab in response to determining the body temperature of the operator is not above the threshold body temperature.

3. The health system of claim 1, wherein the controller is configured to:
receive a second sensor signal indicative of presence of the operator within the cab of the work vehicle; and
output a third control signal to the interlock system indicative of instructions to lock the door of the cab and output a fourth control signal to a sanitation system indicative of instructions to enter a sanitation mode in response to determining the operator is not present within the cab of the work vehicle.

4. The health system of claim 3, wherein the controller is configured to select the sanitation mode from a plurality of candidate sanitation modes based on a time of day, a day of week, a sanitation duration, a sanitation location, a contamination level, one or more environmental factors, or any combination thereof.

5. The health system of claim 4, wherein the plurality of candidate sanitation modes comprises a dry sanitation mode and a wet sanitation mode.

6. The health system of claim 5, wherein the dry sanitation mode comprises heat sterilization, ultra-violet light treatment, or a combination thereof.

7. The health system of claim 5, wherein the wet sanitation mode comprises a pre-rinse cycle, a rinse cycle, a clean cycle, a dry cycle, or a combination thereof.

8. The health system of claim 5, wherein the sanitation system, while in the wet sanitation mode, is configured to output a status signal indicative of a wet sanitation fluid level.

9. The health system of claim 8, wherein the controller is configured to output an information signal to a user interface in response to determining the wet sanitation fluid level is less than or equal to a threshold wet sanitation fluid level.

10. The health system of claim 1, wherein the controller is configured to receive an input signal indicative of an operator ID, wherein the operator ID corresponds to the operator of the work vehicle.

11. The health system of claim 10, wherein the controller is configured to save a record log.

12. The health system of claim 11, wherein the record log comprises the body temperature of the operator, the operator ID, and a state of the interlock system.

13. A health system for a work vehicle, comprising:
a controller comprising a memory and a processor, wherein the controller is configured to:
receive a sensor signal indicative of presence of an operator within a cab of the work vehicle; and
output a first control signal to an interlock system indicative of instructions to lock a door of the cab and output a second control signal to a sanitation system indicative of instructions to enter a sanitation mode in response to determining the operator is not present within the cab of the work vehicle;
wherein the controller is configured to select the sanitation mode from a plurality of candidate sanitation modes based on a time of day, a day of week, a sanitation duration, a sanitation location, a contamination level, one or more environmental factors, or any combination thereof.

14. The health system of claim 13, wherein the plurality of candidate sanitation modes comprises a dry sanitation mode and a wet sanitation mode.

15. The health system of claim 14, wherein the dry sanitation mode comprises heat sterilization, ultra-violet light treatment, or a combination thereof.

16. The health system of claim 14, wherein the wet sanitation mode comprises a pre-rinse cycle, a rinse cycle, a clean cycle, a dry cycle, or a combination thereof.

17. The health system of claim 13, wherein the controller is configured to exit the sanitation mode after a sanitation cycle is complete, and the controller is configured to generate a record log in response to exiting the sanitation mode.

18. A health system for a work vehicle, comprising:
a controller comprising a memory and a processor, wherein the controller is configured to:
receive an input signal indicative of an operator ID, wherein the operator ID corresponds to an operator of the work vehicle;
receive a sensor signal indicative of a absence of the operator from a cab of the work vehicle;
output a first control signal to an interlock system indicative of instructions to lock a door of the cab and output a second control signal to a sanitation system indicative of instructions to enter a sanitation mode in response to determining the operator is absent from the cab of the work vehicle; and
save a record log, wherein the record log comprises the operator ID and the sanitation mode.

19. The health system of claim 18, wherein the controller is configured to select the sanitation mode from a plurality of candidate sanitation modes based on a time of day, a day of week, a sanitation duration, a sanitation location, a contamination level, one or more environmental factors, or any combination thereof.

20. The health system of claim 18, wherein the record log comprises a time of sanitation, a date of sanitation, a duration of sanitation, or any combination thereof.
